(19) United States
(12) Patent Application Publication
(10) Pub. No.: US 2012/0220845 A1
(43) Pub. Date: Aug. 30, 2012

(54) SHOCK OR SEPSIS EARLY DETECTION METHOD AND SYSTEM

(75) Inventor: Shannon Campbell, Boulder, CO (US)

(73) Assignee: Nellcor Puritan Bennett LLC, Boulder, CO (US)

(21) Appl. No.: 13/037,069

(22) Filed: Feb. 28, 2011

(51) Int. Cl. A61B 5/145

(52) U.S. Cl. 600/364

(57) ABSTRACT

According to various embodiments, a medical system and method for early detection of shock and/or sepsis may include devices configured to provide information about carbon dioxide and/or pH levels in a patient's tracheal tissue and in a respiratory path. Specifically, a relationship between carbon dioxide and/or pH in the tracheal tissue vs. respiratory gases (e.g., lung gases) may be used to determine if a patient is at risk of developing shock or sepsis. In certain embodiments, if carbon dioxide in the tracheal tissue deviates from end-tidal carbon dioxide, a patient may be experiencing early stages of hypoperfusion associated with shock and/or sepsis.

### BACKGROUND

The present disclosure relates generally to medical devices and, more particularly, to airways devices that are configured to detect sepsis or shock.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare, monitor the progress of patient care, and detect changing conditions that potentially need attention. As a result, such monitoring devices have become an indispensable part of modem medicine.

Physiological characteristics that physicians may often desire to monitor include constituents of the blood and tissue, such as oxygen and carbon dioxide. For example, abnormal levels of carbon dioxide in the blood or tissue may be related to poor perfusion. Thus, assessment of carbon dioxide levels may be useful for diagnosing a variety of clinical states related to poor perfusion. Carbon dioxide and other blood constituents may be directly measured by taking a blood sample, or may be measured indirectly by assessing the concentration of those constituents in the tissue or respiratory gases. For example, carbon dioxide in the bloodstream equilibrates rapidly with carbon dioxide in the lungs, and the partial pressure of the carbon dioxide in the lungs approaches the amount in the blood during each breath. Accordingly, physicians often monitor respiratory gases during breathing in order to estimate the carbon dioxide levels in the blood. In certain instances, it may be advantageous to assess carbon dioxide in respiratory gases that are flowing through airway devices.

While detection of abnormal levels of carbon dioxide in the lungs may be useful in identifying patients with hypoperfusion, low perfusion levels may be associated with a number of complex and overlapping clinical conditions. For example, shock, sepsis, and cardiac failure may all present with systemic hypoperfusion. Accordingly, hypoperfusion as determined by sampling respiratory gases may be insufficient to distinguish between shock and respiratory failure and/or insufficient ventilation (e.g., VQ mismatch) and, thus, may yield an incomplete diagnosis. Physicians may also test for the presence of shock-associated biomarkers to distinguish between shock and other conditions. However, biomarkers tests may have slow turnaround times. Further, certain biomarkers of shock may not emerge until the disease has progressed to a point where certain treatments may be less effective.

### BRIEF DESCRIPTION OF THE DRAWINGS

Advantages of the disclosure may become apparent upon reading the following detailed description and upon reference to the drawings in which:

FIG. 1 illustrates a system for early detection of shock and/or sepsis including a tracheal tube with a tracheal tissue sensor and respiratory gas sensor according to embodiments of the present techniques;

FIG. 2 is a perspective view of a tracheal tube with a deployable tracheal tissue sensor that may be used in conjunction with the system of FIG. 1;

FIG. 3 is a perspective view of a tracheal tube with a deployable tracheal tissue sensor associated with an inflatable cuff that may be used in conjunction with the system of FIG. 1;

FIG. 4 is a flow diagram of an exemplary method for detecting shock and/or sepsis;

FIG. 5 is an example of a trend view of tracheal tissue carbon dioxide and end-tidal carbon dioxide for a patient who is developing shock and/or sepsis, as detectable via the present techniques; and

FIG. 6 is a flow diagram of an exemplary method for administering treatment for a patient who has been diagnosed with a shock-related syndrome.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Physiological shock may result from any serious assault on the body's homeostatic mechanisms, whether from hemorrhage, trauma, burn injury, myocardial infarction or sepsis. Shock may involve widespread hypoperfusion at the tissue level, due to reduction of blood volume, reduction of cardiac output or redistribution of effective circulation. While sepsis also involves a systemic host involvement, sepsis generally refers to a host defense response that accompanies a microbial invasion. The timely detection of shock and/or sepsis may be complex. For example, septic shock is often the end result of progression from an infection to progressive states of the inflammatory response syndrome, sepsis, severe sepsis, and finally septic shock. While a vast majority of patients with infection do not progress to septic shock, identification of those patients who are progressing through the stages of septic shock is poorly defined, and reliable technologies for detection of the advancement of such states are not in place. Because routine blood testing and even continuous vital sign measurements may not always detect the pre-shock state, specialized blood tests and biomarker profiles specifically developed to detect the pre-septic shock state have been developed. However, specific blood test and profiles suffer from a lack of specificity, in part because of the variable response of patients to disease progression.

Provided herein are medical devices for the early detection of shock and/or sepsis. In certain embodiments, early stages of shock may be characterized by changes in perfusion of the tracheal tissue. At the onset of shock and/or sepsis (or related syndromes), the body shunts blood flow away from secondary tissues such as the tracheal mucosa, and these tissues undergo hypoperfusion. During hypoperfusion, the cells shift from aerobic metabolism to anaerobic metabolism, which results in an increase in the production of carbon dioxide and lactic acid. Accordingly, a gap may develop between the pH and carbon dioxide content of the tracheal mucosa and the arterial blood. By monitoring the appearance and size of this gap, shock or sepsis may be detected at an early stage before the disease has progressed to later stages that may involve more clinical complications. While assessment of carbon dioxide levels and pH of the tracheal mucosa may be accomplished with sensors that contact this tissue, in certain embodiments, monitoring arterial blood may involve invasive techniques for direct measurement. However, as provided herein, end-tidal carbon dioxide during respiration may be used as a surrogate for arterial carbon dioxide content.

In certain embodiments, the present techniques may be used in conjunction with any appropriate medical device, including a feeding tube, an endotracheal tube, a tracheostomy tube, a bronchocatheter, a circuit, an airway accessory, a connector, an adapter, a filter, a humidifier, a nebulizer, nasal cannula, or a laryngeal mask. The present techniques may also be used to monitor any patient benefiting from mechanical ventilation. Further, the devices and techniques provided herein may be used to monitor a human patient, such as a trauma victim, an intubated patient, a patient with a tracheostomy, an anesthetized patient, a cardiac arrest victim, a patient suffering from airway obstruction, or a patient suffering from respiratory failure.

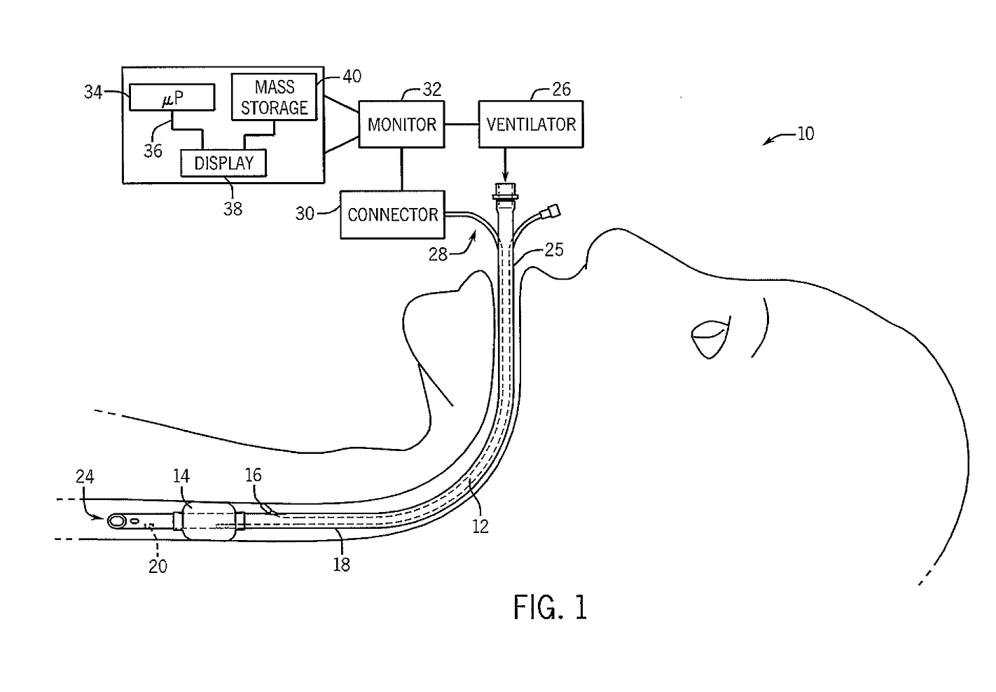

FIG. 1 shows an exemplary tracheal tube system 10 including a tracheal tube 12 that has been inserted into the trachea of a patient. The system 10 includes a tracheal tube 12 with an inflatable balloon cuff 14 that may be inflated to form a seal against the tracheal walls. The tracheal tube 12 may also include a tracheal tissue sensor 16 that is associated with an exterior surface 18 of the tube 12. In addition, the system 10 may include a respiratory sensor 20 for sampling respiratory gases during a breathing cycle. As shown, the respiratory sensor 20 may be associated with a distal end 24 of the tube 12 and, in certain embodiments, may be disposed on an interior surface of the tracheal tube 12 (e.g., may be adhered to, embedded in, or otherwise associated with the interior flow path of the tracheal tube). In other embodiments, the respiratory sensor 20 may be associated with the distal end 24 in any suitable arrangement for sampling respiratory gases and may, in particular embodiments, be associated with an end region or exterior surface of the distal end 24. However, in certain embodiments, the respiratory sensor 20 may be positioned closer to a proximal end 25 or at any appropriate location along a breathing circuit and may be coupled to upstream tubing or connectors that couple the tracheal tube 12 to a ventilator 26. The tracheal tissue sensor 16 and the respiratory sensor 20 may be coupled by one or more cables 28 to a connector 30 that may be coupled to a medical device, such as the ventilator 26 or a monitor 32, which in turn may be coupled to central medical stations (e.g., a nurse's station or central hospital station).

As noted, the system 10 may also include devices that facilitate positive pressure ventilation of a patient, such as the ventilator 26, which may include any ventilator, such as those available from Nellcor Puritan Bennett LLC. The ventilator 26 may be coupled to the monitor 32, which may be configured to implement embodiments of the present disclosure to detect shock and/or sepsis based upon certain parameters in the tracheal tissue relative to respiratory gases. It should be understood that the monitor 32 may be a stand-alone device or may, in embodiments, be integrated into a single device with another medical device, such as the ventilator 26.

The monitor 32 may include processing circuitry, such as a microprocessor 34 coupled to an internal bus 36 and a display 38. In an embodiment, the monitor 32 may be configured to communicate with the tracheal tube 12, for example via connector 30, to obtain signals from the tracheal tissue sensor 16 and/or the respiratory sensor 20. In certain embodiments, the connector 30 may also provide calibration information for the tube 12. The information may then be stored in a mass storage device 40, such as RAM, PROM, optical storage devices, flash memory devices, hardware storage devices, magnetic storage devices, or any suitable computer-readable storage medium. The information may be accessed and operated upon according to microprocessor 34 instructions. In certain embodiments, calibration information may be used in calculations for determining a presence or likelihood of shock and/or sepsis based on tracheal tissue and respiratory gas parameters. The monitor 32 may be configured to provide indications of a risk or presence of shock and/or sepsis, such as an audio, visual or other indication, or may be configured to communicate the information to another device. In an embodiment, based at least in part upon the received signals from the tracheal tissue sensor 16 and/or the respiratory sensor 20, microprocessor 34 may calculate a difference between carbon dioxide and/or pH of the tracheal tissue and end tidal carbon dioxide (or expiratory pH) using various algorithms. Alternatively, the algorithms may be configured to use direct measurements of arterial blood parameters (e.g., carbon dioxide or pH) in the comparison. The algorithms may employ additional correction coefficients. In general, such algorithms will be stored in non-transitory computer media (e.g., memory) and executed by the processing circuitry as described below.

The tracheal tissue sensor 16 and the respiratory sensor 20 as provided herein may include any appropriate sensor or sensor element for assessing carbon dioxide or pH, either in the tissue or in respiratory gases, including chemical, electrical, optical, non-optical, quantum-restricted, electrochemical, enzymatic, spectrophotometric, fluorescent, or chemiluminescent indicators or transducers. In certain embodiments, the tracheal tissue sensor 16 and/or the respiratory sensor 20 may include optical components, e.g., an emitter and detector pair that may be of any suitable type. For example, the emitter may be one or more light emitting diodes adapted to transmit one or more wavelengths of light in the green or red to infrared range, and the detector may be one or more photodetectors selected to receive light in the range or ranges emitted from the emitter. The wavelength may be selected according to considerations for penetration distance. For example, green light may be suitable for shallower penetration into the tissue. Alternatively, an emitter may also be a laser diode or a vertical cavity surface emitting laser (VCSEL). An emitter and detector may also include optical fiber sensing components. An emitter may include a broadband or “white light” source, in which case the detector could include any of a variety of elements for selecting specific wavelengths, for example, reflective or refractive elements or interferometers. These kinds of emitters and/or detectors would typically be coupled to the rigid or rigidified sensor via fiber optics. Alternatively, the tracheal tissue sensor 16 and/or the respiratory sensor 20 may sense light detected through the respiratory gas at a different wavelength from the light emitted into the respiratory gas. Such sensors may be adapted to sense fluorescence, phosphorescence, Raman scattering, Rayleigh scattering and multi-photon events or photoacoustic effects. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the ultrasound, radio, microwave, millimeter wave, infrared, visible, ultraviolet, gamma ray or X-ray spectra.

The tracheal tissue sensor 16 and/or the respiratory sensor 20 may be an electrochemical transducer, which may be adapted to detect and measure changes in ambient chemical parameters induced by the presence of critical amounts of carbon dioxide. In one embodiment, the sensing component 10 may include a sensor that employs cyclic voltammetry for carbon dioxide detection. Such sensors are available from Giner, Inc., Newton, Mass. For example, the tracheal tissue sensor 16 and/or the respiratory sensor 20 may be a thick film catalyst sensor utilizing a proton exchange membrane. Such a sensing component 10 may include thick film screen printed electrodes and an electrochemically reversible metal oxide catalysts. Appropriate catalysts include MO, M₂O₃, MO₂, where M is a metal that is any suitable metal, including platinum, ruthenium, or iridium. Generally, such sensors operate by sensing chemical reactions caused by proton dissociation from water in which carbon dioxide is dissolved. Dissociated water protons may electrochemically reduce a metal oxide layer of the sensor. The electrochemical reduction of the metal oxide will result in generation of an electrical current, which varies in response to the degree of electrochemical reduction.

In another embodiment, the tracheal tissue sensor 16 and/or the respiratory sensor 20 may include quantum-restricted components, including carbon nanotubes, buckeyballs, or quantum dots. Generally, quantum-restricted components may be coated or otherwise modified with a compound that is sensitive to the respiratory gas of interest. Interaction of the respiratory gas with the compound may affect the electrical, optical, thermal, or physical properties of the quantum-restricted components such that a signal may result. In one such example, carbon nanotubes may be coated with a carbon dioxide-sensitive compound or polymer, such as a polyethyleneimine and starch polymer. Carbon dioxide may combine with primary and tertiary amines in the polyethyleneimine and starch polymer coating to form carbamates. The chemical reaction alters the charge transfer to the carbon nanotubes and results in an electrical signal. Other suitable polymer coatings may be adapted to sense other respiratory gases of interest, such as oxygen or carbon monoxide. In other embodiments, the quantum-restricted component may include a binding molecule, such as a receptor or an enzyme that is specific for the respiratory gas of interest. One such molecule may include carbonic anhydrase. Binding of the respiratory gas to its receptor may affect a downstream response that may result in a change in the electrical properties of a quantum-restricted component.

The tracheal tissue sensor 16 and/or the respiratory sensor 20 may also include a semi-conductive sensing element, such as a field-effect transistor (FET) or an ion-sensitive field-effect transistor (ISFET). An ISFET may include a silicon dioxide gate for a pH selective membrane. Such a sensor may be adapted to sense downstream changes in hydrogen ion concentration in response to changes in carbon dioxide or other respiratory gas concentrations. In certain embodiments, the semi-conductive sensing element may be a film.

Alternatively, the tracheal tissue sensor 16 and/or the respiratory sensor 20 may include any indicator that is sensitive to the presence of carbon dioxide and that is capable of being calibrated to give a response signal corresponding to a given predetermined concentration of carbon dioxide. The signal may be visual, e.g., a change in color, or electrical. Indicators that provide a color change in a presence of carbon dioxide may include chromogenic pH-sensitive indicators and oxidation/reduction indicators. A chemical indicator may be used in conjunction with an electrical or electronic device that is adapted to detect and measure changes in the ambient chemical parameters induced by the presence of critical amounts of carbon dioxide. For example, optical fiber carbon dioxide sensing components may be used to convert a change in a chemical indicator to a quantitative measurement of carbon dioxide in the sample. Generally, such sensing components operate by directing light of a predetermined wavelength from an external source through the optical fiber to impinge the chemical indicator. The intensity of the emitted fluorescent light returning along the fiber is directly related to the concentration of carbon dioxide in the sample, as a result of the pH-sensitive indicator material present at the fiber tip (i.e., the pH of the indicator solution is directly related to carbon dioxide concentration, as a result of carbonic acid formation). The emitted light is carried by the optical fiber to a device where it is detected and converted electronically to a carbon dioxide concentration value. The tracheal tissue sensor 16 and/or the respiratory sensor 20 may additionally have a reference dye present in the indicator composition. The intensity of the light emitted from the reference dye may be used to compensate, via ratioing, the signal obtained from the indicator. Other components may be incorporated into the indicator composition including surfactants, antioxidants and ultraviolet stabilizers. The tracheal tissue sensor 16 and/or the respiratory sensor 20 may be formed from any appropriate substrate. The tracheal tissue sensor 16 and/or the respiratory sensor 20 may also include a borosilicate sensing element such as those discussed in the U.S. patent application Ser. No. 11/526,393, filed on Sep. 25, 2006, which is hereby incorporated by reference in its entirety herein for all purposes.

In particular embodiments, the system 10 may be used with a tracheal tube that includes sensor configured to provide information about tracheal tissue (e.g., tracheal tissue sensor 16) as well as a sensor configured to provide information about the carbon dioxide content of respiratory gases throughout a breathing cycle (e.g., respiratory sensor 20). While the respiratory sensor 20 may be directly associated with the tube 12 or any part of the breathing circuit to come into contact with and sense parameters of respiratory gases, providing a sensor that is configured to contact a tracheal mucosa may be more complex. As provided, in certain embodiments, a tracheal tube 12 having a mechanically deployable tracheal tissue sensor 16, may be used in conjunction with the system 10, such as those discussed in the U.S. patent application Ser. Nos. 12/713,323 and 12/713,351, filed on Feb. 26, 2010, which are hereby incorporated by reference in their entirety herein for all purposes.

FIG. 2 is an elevational view of an implementation in which the tracheal tube 12 features a mechanically deployable member 50 on which the tracheal tissue sensor 16 is disposed. The tracheal tissue sensor 16 may be coupled to the cable 28 and the connector 30 (see FIG. 1). In the illustrated embodiment, the proximal end 25 of the tracheal tube 12 includes a connecting feature 51 that may be coupled to additional tubing and attached to a mechanical ventilator during operation. The distal end 24 terminates in an opening 52 and may be placed in a trachea of a patient during operation to maintain airflow to and from the lungs. A Murphy's eye 54 may be located on the tube 12 close to the distal end 24 to prevent airway occlusion when the tube 12 is improperly placed within the trachea. The cuff 14 that may be inflated to seal against the walls of a body cavity (e.g., the trachea) and may be inflated via an inflation lumen 56 terminating in an inflation tube 58 connected to a fixture 60.

The tube 12 and the cuff 14 may be formed from materials having desirable mechanical properties (e.g., puncture resistance, pin hole resistance, tensile strength, and so forth) and desirable chemical properties (e.g., biocompatibility). In one embodiment, the walls of the cuff 14 may be made of a polyurethane (e.g., Dow Pellethane® 2363-80A) having suitable mechanical and chemical properties. In other embodiments, the walls of the cuff 14 may be made of a suitable polyvinyl chloride (PVC). In certain embodiments, the cuff 14 may be generally sized and shaped as a high volume, low pressure cuff that may be designed to be inflated to pressures between about 15 cm and 30 cm of water. The cuff 14 may be any of a variety of suitable cuffs, such as a tapered cuff or a non-tapered cuff.

The tracheal tissue sensor 16 mounted on a tip of the deployment member 50 (e.g., polymeric tube, plastic support, etc.) is configured to be mechanically deployed in the trachea during use. In one embodiment, a lumen 62 is disposed along the tracheal tube 12 from the proximal end 24 to a location above the cuff 14. The deployment member 50 is adapted to be partially disposed in the lumen 62 during intubation. That is, while the patient is being intubated or extubated, the sensor 16 is configured to rest in a recess 63 disposed in the tracheal tube 12, and a first portion 64 of the deployment member 50 is adapted to terminate outside of the lumen 62. After intubation, the deployment member 50 may be slid lengthwise along the tracheal tube 12, thus deploying the tracheal tissue sensor 16. The tracheal tissue sensor 16 may be deployed to a position that abuts the tracheal wall or is in close proximity to the tracheal mucosa. Furthermore, in some embodiments, the recess 63 may include a ramp portion that is adapted to guide the tracheal tissue sensor 16 toward the tracheal wall as the tracheal tissue sensor 16 is deployed. Nevertheless, during deployment, tracheal tissue sensor 16 is configured to measure the presence or level of one or more blood gases and/or blood analytes. After deployment, the tracheal tissue sensor 16 may be mechanically withdrawn from its deployment position and returned to the recess 63.

It should be noted that the deployment member 50 may be any of a variety of suitable deployment apparatuses with a variety of functionalities. For instance, the deployment member 50 may be a polymeric tube that is configured to undergo a variety of tensile and compressive forces (e.g., as the deployment member is slid along the length of the tracheal tube). The deployment member 50 may be further adapted to encase one or more conductors that terminate in the tracheal tissue sensor 16 or may be coupled to the conductors. For example, the conductors terminating in the tracheal tissue sensor 16 may be secured to a support that facilitates the deployment of the tracheal tissue sensor 16. To that end, the deployment member 50 may function to encase one or more conductors, to provide the structure necessary to withstand tensile and compressive forces, and to facilitate the deployment of the tracheal tissue sensor 16.

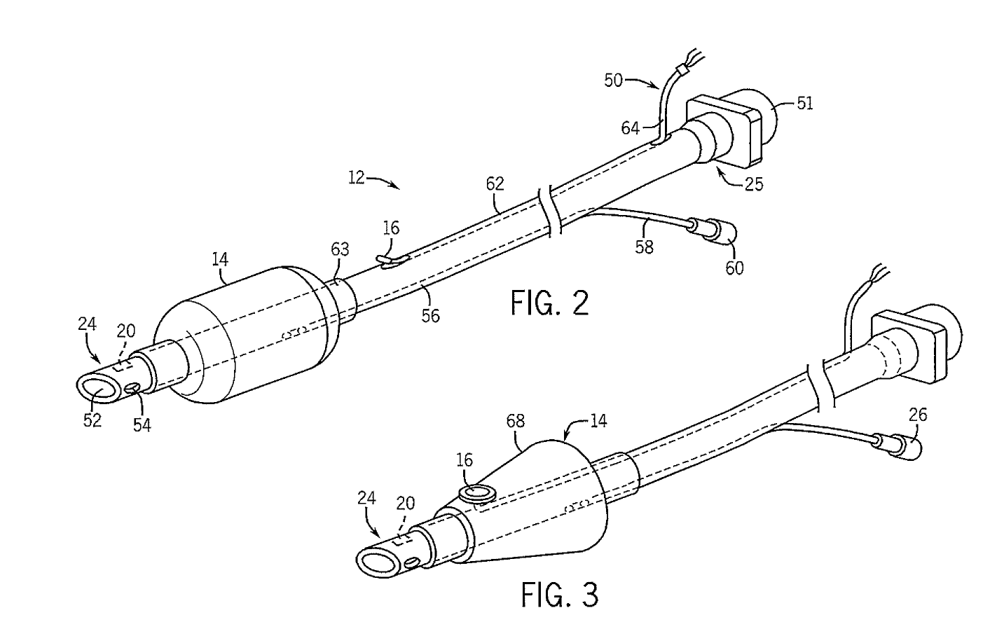

In another embodiment, the tracheal tissue sensor 16 may be configured to contact the tracheal wall via inflation of an inflatable member (e.g., the cuff 14). As the tracheal tube 12 is inserted, the cuff 14 is in a deflated stated. After insertion, the cuff 14 may be inflated to contact the tracheal walls. In particular embodiments, the inflatable member may be a sealing cuff configured to provide a seal between the lower airway and the upper airway. In other embodiments, the tracheal tissue sensor 16 may be disposed on a secondary cuff that is located distally of the sealing cuff 14. In certain embodiments, the tracheal tissue sensor 16 may be disposed on a non-sealing portion of the cuff 14. That is, the sensor may be located anywhere on the cuff 14 that is not configured to provide the seal desired between the body of the tube and the body tissues (e.g., directly contact the body cavity, such as the tracheal wall), during inflation of the cuff 14. For instance, in one embodiment, the sensor may be positioned at the tapered end 68 of the cuff as shown in FIG. 3. As depicted, the cuff 14 tapers towards the distal end 24 of the tube 12. However, in other embodiments, the cuff 14 may be reversed in orientation with respect to the tracheal tube 12. That is, the cuff 14 may taper towards the proximal end 25. In particular, the placement of the tracheal tissue sensor 16 facilitates contact with the tracheal wall without adversely affecting the seal between the cuff and the tracheal wall.

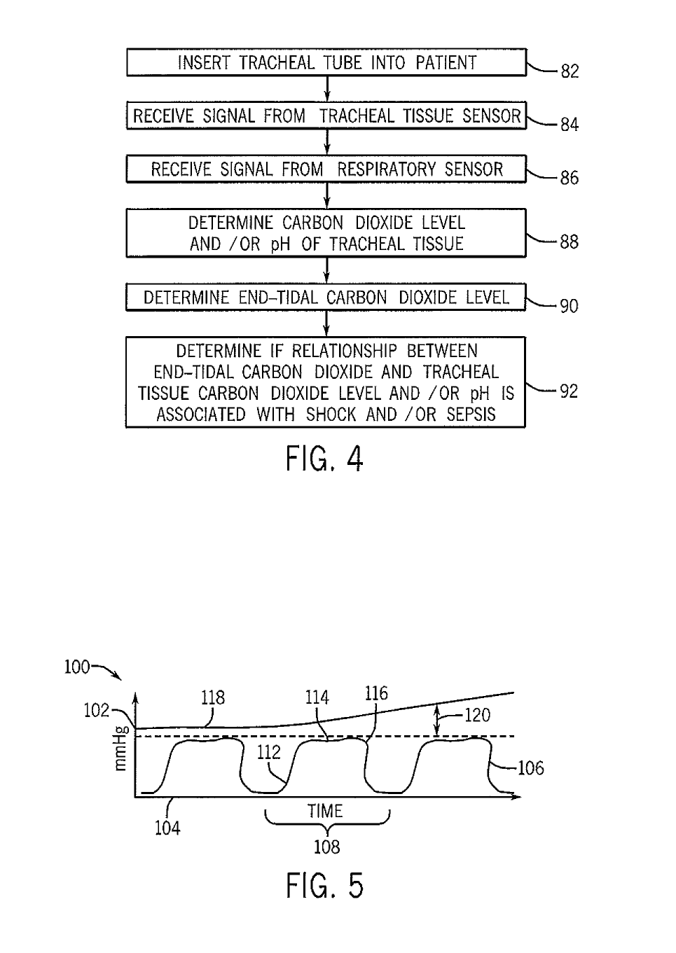

FIG. 4 is an exemplary process flow diagram illustrating a method for detection of shock and/or sepsis. The method is generally indicated by reference number 80 and includes various steps or actions represented by blocks. It should be noted that the method 80 may be performed as an automated or semiautomated procedure by a system, such as system 10. Further, certain steps or portions of the method may be performed by separate devices. For example, a portion of the method 80 may be performed by a clinician, while a second portion of the method 80 may be performed by a monitor 32. In embodiments, the method 60 may be performed continuously or intermittently for long-term patient monitoring or at any appropriate interval depending on the particular situation of the intubated patient.

According to a presently contemplated embodiment, the method 80 begins with insertion of the tracheal tube 12 into the patient at step 82. The insertion of the tracheal tube 12 may be accompanied by the deployment or positioning of the tracheal tissue sensor 16. In addition, if the respiratory sensor 20 is associated with an upstream portion of the respiratory circuit, step 82 may include coupling the respiratory sensor 20 to the respiratory circuit. After coupling the sensors (e.g., sensor 16 and 20) to the patient, the sensors may start collecting measurement data. At step 84, a signal from the tracheal tissue sensor 16 is received by the monitor 32 and at step 86 a signal from the respiratory sensor 20 is received by the monitor 32. The monitor 32 may process the signals in any appropriate fashion to determine the carbon dioxide level or pH of the tracheal tissue at step 88 and the end-tidal carbon dioxide level at step 90 based on the signal from the tracheal tissue sensor 16 and the respiratory sensor 20, respectively. Based on the relationship between the tracheal carbon dioxide and/or pH and the end-tidal carbon dioxide, the monitor may assess if the relationship is indicative of shock and/or sepsis at step 92.

The carbon dioxide levels measured by the present techniques may be analyzed via a graphical capnogram that may illustrate the carbon dioxide levels between the tracheal tissue and the end-tidal carbon dioxide. FIG. 5 is an example of a hypothetical capnogram 100 with tracheal tissue carbon dioxide information, which shows carbon dioxide concentration (on y-axis 102) over time x-axis 104). The output of the respiratory sensor 20, shown as graphed line 106, rises as falls over the course of a breath cycle 108. During inhalation, the carbon dioxide levels falls as oxygen is taken into (or, in the case of mechanical ventilation, pushed into) the lungs. As exhalation beings, an expiratory upstroke 112 is observed, followed by an expiratory plateau 114 and an end-tidal carbon dioxide peak 116. End-tidal carbon dioxide is the partial pressure or maximal concentration of carbon dioxide at the end of an exhaled breath, which is expressed as a percentage of CO₂ or mmHg. The normal values are 5% to 6% CO2, which is equivalent to 35-45 mmHg. The end-tidal carbon dioxide may reflect cardiac output and pulmonary blood flow as the gas is transported by the venous system to the right side of the heart and then pumped to the lungs by the right ventricles. Because carbon dioxide diffuses out of the lungs into the exhaled air, the partial pressure or maximal concentration of carbon dioxide at the end of exhalation may be used as a surrogate for arterial carbon dioxide.

The carbon dioxide levels as may be assessed by a tracheal tissue sensor 16 are shown in line 118. During normal tissue perfusion, the tracheal tissue carbon dioxide may be substantially the same as or, as shown, may differ by a small but generally constant amount from the end-tidal carbon dioxide 116. In certain embodiments, this difference may be accounted for in determining a relationship between the tracheal tissue carbon dioxide and the end-tidal carbon dioxide. During the early stages of shock and/or sepsis, as the tracheal tissue undergoes hypoperfusion, the carbon dioxide levels in the tracheal tissue go up and the gap 120 between the end-tidal carbon dioxide 116 increases. The degree or risk of shock and/or sepsis may be calculated from the size of the gap 120, or may depend on whether the gap 120 is larger than a predetermined threshold. In one embodiment, if the gap 120 is larger than 10, 15, 20, 25, or 30 mm Hg, an indication of shock and/or sepsis may be triggered (e.g., an alarm). An indication related to a severity of the gap 120 may be provided, such as a red, yellow, or green indicator for different severity levels. In another embodiment, the rate of change of the gap 120 may be assessed in determining the risk of shock and/or sepsis for the patient. In one embodiment, the end-tidal carbon dioxide is examined over a predetermined time window to assess changes. Various signal processing techniques may be employed, such as periodically sampling signals from the sensor, analyzing the signals to determine when end-tidal carbon dioxide levels are reached, then storing and low pass filtering the retained values over several cycles to avoid noise, spikes, and so forth. For example, the end-tidal carbon dioxide may be assessed as a rolling average of the previous five breaths. Further, it is contemplated that a display of the tracheal tissue carbon dioxide as compared to the respiratory carbon dioxide over time may provide a visual representation of an increase in the tracheal tissue carbon dioxide and may provide visual information to clinicians regarding a change in a patient's clinical condition. It should be noted, however, that the present techniques for assessing the onset or progression of shock and/or sepsis may be performed, and output provided to caregivers without actual representation of the parameter levels (e.g., graphically).

Although the depicted embodiment shows tracheal carbon dioxide levels, a similar assessment may be made by examining tracheal tissue pH and its relationship to end-tidal carbon dioxide. Generally, tracheal tissue pH decreases as lactic acid build up in the cells during hypoperfusion. Accordingly, relative to a baseline level, a change in pH in a tracheal mucosal tissue may be compared to end-tidal carbon dioxide. In addition, in certain embodiments, the carbon dioxide and/or pH of the tracheal tissue may be compared with arterial blood parameters.

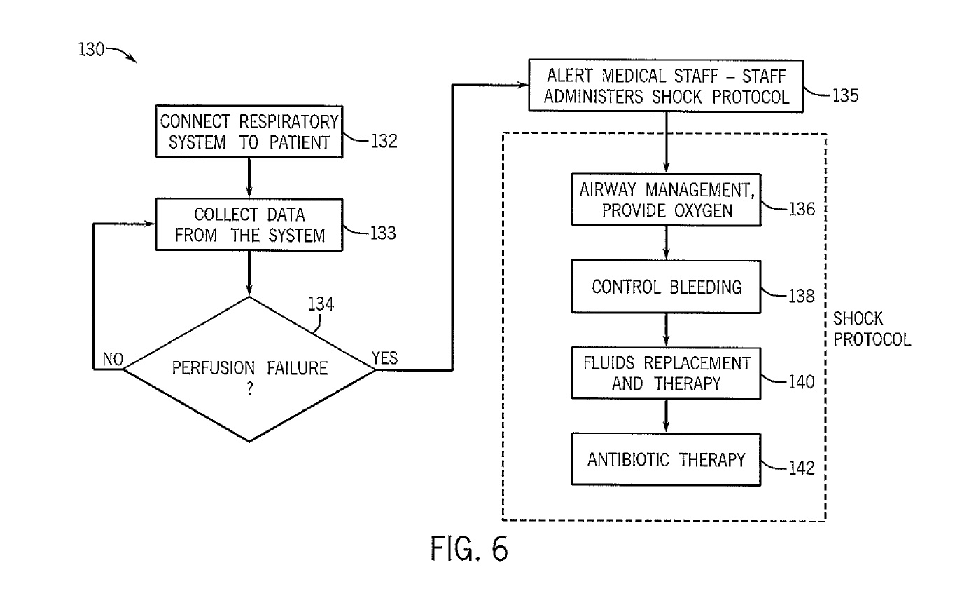

FIG. 6 shows a detailed example of an embodiment of a treatment protocol method 130 that may be triggered by the early detection of shock. Initially, a respiratory system 10 may be connected to a subject at step 132. The system may collect data from the system 10 at step 133. Based on a relationship between tracheal tissue parameters, such as carbon dioxide and/or pH, and the end-tidal carbon dioxide in the respiratory circuit, the system 10 may determine if the patient is in an early stage of shock. If no perfusion failure is detected, then the tissue continues to be monitored. If perfusion failure is detected, then medical staff may be alerted to subsequently administer a shock protocol at step 135. In the present embodiment, the first step of shock protocol may be to provide oxygen via airway management at step 136. Subsequently, efforts are made control and limit the patient's bleeding at step 138. The shock protocol may also include fluid replacement and therapy and/or antibiotic therapy (steps 140, 142). As will be appreciated, the steps that may be performed during a shock treatment procedure may vary depending upon the stage of the patient's shock and guidelines provided by different healthcare institutions. One embodiment may include administration of Early Goal Directed Therapy (EGDT).

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A method, comprising:
using processing circuitry:
receiving a first signal from a first sensor in contact with a tracheal tissue of a patient, wherein the first signal is representative of a level of carbon dioxide or a pH of the tracheal tissue;
receiving a second signal from a second sensor, wherein the second signal is representative of an end-tidal level of carbon dioxide during respiration of the patient or an arterial level of carbon dioxide or an arterial pH; and
determining whether the patient is at risk for developing shock or sepsis based at least in part on a relationship between the level of carbon dioxide or pH of the tracheal tissue and the end-tidal level of carbon dioxide or the arterial level of carbon dioxide or the arterial pH.

2. The method of claim 1, comprising providing a user-perceptible indication related to a risk of developing shock or sepsis.

3. The method of claim 1, comprising displaying a comparison of the level of carbon dioxide in the tracheal tissue and the end-tidal level of carbon dioxide over time.

4. The method of claim 1, wherein the relationship comprises a difference between the level of carbon dioxide in the tracheal tissue and the end-tidal level of carbon dioxide.

5. The method of claim 4, comprising triggering an alarm when the difference between the level of carbon dioxide in the tracheal tissue and the end-tidal level of carbon dioxide is greater than a threshold.

6. The method of claim 1, wherein the relationship comprises a rate of change in a difference between the level of carbon dioxide in the tracheal tissue and the end-tidal level of carbon dioxide.

7. The method of claim 1, comprising providing a user perceptible indication that shock or sepsis therapy should be started if the patient is at risk for developing shock or sepsis.

8. A monitor, comprising:
processing circuitry configured to execute instructions for:
receiving a first signal from a first carbon dioxide sensor in contact with a tracheal tissue of an intubated patient;
determining a level of carbon dioxide in the tracheal tissue based on the first signal;
receiving a second signal from a second carbon dioxide sensor in fluid communication with a respiratory circuit of the intubated patient;
determining an end-tidal level of carbon dioxide during respiration based on the second signal; and
determining a clinical condition of the patient based at least in part on a relationship between the level of carbon dioxide in the tracheal tissue and the end-tidal level of carbon dioxide.

9. The monitor of claim 8, wherein the processor comprises instructions to trigger an alarm when a difference between the level of carbon dioxide in the tracheal tissue and the end-tidal level of carbon dioxide is greater than a predetermined level.

10. The monitor of claim 8, comprising a display for displaying a user perceptible indication related to the clinical condition of the patient.

11. The monitor of claim 10, wherein the clinical condition of the patient comprises shock or sepsis.

12. The monitor of claim 8, wherein the relationship comprises a difference between the level of carbon dioxide in the tracheal tissue and the end-tidal level of carbon dioxide.

13. The monitor of claim 8, wherein the relationship comprises a rate of change of a difference between the level of carbon dioxide in the tracheal tissue and the end-tidal level of carbon dioxide.

14. The monitor of claim 8, wherein the processor comprises instructions for correcting for a higher baseline level of carbon dioxide in the tracheal tissue relative to the end-tidal level of carbon dioxide.

15. The monitor of claim 8, wherein determining the end-tidal level of carbon dioxide during respiration comprises determining an average end-tidal carbon dioxide level over a predetermined number of breaths.

16. The monitor of claim 8, wherein processor comprises instructions for receiving a third signal related to a pH of the tracheal tissue, and wherein between the level of carbon dioxide in the tracheal tissue and the end-tidal level of carbon dioxide is based at least in part on the pH of the tracheal tissue.

17. A respiratory system, comprising:
a tracheal tube comprising a passageway for conveying gases to a patient;
a first sensor associated with an exterior of the tracheal tube and configured to contact a tracheal tissue when the tracheal tube is inserted in the patient;
a second sensor in fluid communication with the passageway; and
a monitor operatively coupled to the first sensor and the second sensor, the monitor comprising:
processing circuitry configured to execute instructions for:
receiving a first signal from the first sensor, wherein the first signal is representative of a level of carbon dioxide or a pH of the tracheal tissue;
receiving a second signal from the second sensor, wherein the second signal is representative of an end-tidal level of carbon dioxide during respiration of the patient; and
determining whether the patient is at risk for developing shock or sepsis based at least in part on a relationship between the level of carbon dioxide or pH of the tracheal tissue and the end-tidal level of carbon dioxide.

18. The respiratory system of claim 17, wherein the first sensor is disposed on a non-sealing portion of an inflatable balloon cuff associated with the tracheal tube.

19. The respiratory system of claim 17, wherein the first sensor is disposed on a deployable member associated with the exterior of the tracheal tube.

20. The respiratory system of claim 17, wherein the exterior of the tracheal tube comprises a recess adapted to accommodate the deployable member in an undeployed state.
